(19) United States
(12) Patent Application Publication
(10) Pub. No.: US 2026/0014355 A1
(43) Pub. Date: Jan. 15, 2026

(54) Catheter Clips and Catheter Insertion Assemblies Therewith

(71) Applicant: Bard Access Systems, Inc., Salt Lake City, UT (US)

(72) Inventor: Weston Finch Harding, Lehi, UT (US)

(21) Appl. No.: 18/769,200

(22) Filed: Jul. 10, 2024

(51) Int. Cl. A61M 25/02

(52) U.S. Cl. CPC A61M 25/02; A61M 2025/024

(57) ABSTRACT

Catheter clips and catheter insertion assemblies including such catheter clips facilitate sterile catheterization technique, thereby reducing the risk of catheter-related bloodstream infections and increasing positive patient-centered outcomes. In an example, a catheter clip can include a pair of lever arms mated to each other. Each lever arm of the pair of lever arms can be bilaterally asymmetric but identical to the other lever arm of the pair of lever arms. An elastomeric band can be over a jaw end portion of the catheter clip. A catheter retainer can be coupled to a handle end portion of the catheter clip. The catheter retainer can be configured to hold a catheter connector of a catheter such that at least a proximal portion of the catheter remains in a sterile field when the catheter clip is clipped onto a sterile drape that establishes at least a portion of the sterile field.

### BACKGROUND

Catheter-related bloodstream infections (“CRBSIs”) lead to significant health and economic burdens, so sterile catheterization technique with, for example, central venous catheters (“CVCs”), is vital for preventing CRBSIs.

Disclosed herein are catheter clips and catheter insertion assemblies including such catheter clips that facilitate sterile catheterization technique.

### SUMMARY

Disclosed herein is a catheter clip including, in some embodiments, a pair of lever arms mated to each other, an elastomeric band, and a catheter retainer. Each lever arm of the pair of lever arms is bilaterally asymmetric but identical to the other lever arm of the pair of lever arms. The elastomeric band is over a jaw end portion of the catheter clip. The catheter retainer is coupled to a handle end portion of the catheter clip. The catheter retainer is configured to hold a catheter connector of a catheter such that at least a proximal portion of the catheter remains in a sterile field when the catheter clip is clipped onto a sterile drape that establishes at least a portion of the sterile field.

In some embodiments, the catheter clip further includes a shared pair of fulcrum points between mating faces of the pair of lever arms. The pair of fulcrum points is defined by a discontinuous axle distributed in a discontinuous bearing over a width of the catheter clip.

In some embodiments, each lever arm of the pair of lever arms includes both an axle extension tab and a bearing extension tab extending from a mating face thereof. The axle extension tab and the bearing extension tab establish at least a portion of bilateral asymmetry in the catheter clip.

In some embodiments, an axle portion of the axle extension tab of each lever arm of the pair of lever arms is seated in an opposing bearing portion of the bearing extension tab of the other lever arm of the pair of lever arms, thereby establishing the discontinuous axle distributed in the discontinuous bearing over the width of the catheter clip.

In some embodiments, the bearing extension tab of each lever arm of the pair of lever arms includes one or two side posts lateral of the bearing portion of the bearing extension tab. The one-or-two side posts are configured to keep the axle portion of the axle extension tab seated in the bearing portion of the bearing extension tab.

In some embodiments, an external face of each lever arm of the pair of lever arms includes a transverse channel longitudinally located between the pair of fulcrum points and a pair of elastomeric clip tips, which clip tips respectively extend from the pair of lever arms in the jaw end portion of the catheter clip. The channel of each lever arm of the pair of lever arms is configured to accommodate the elastomeric band therein.

In some embodiments, the jaw end portion of the catheter clip includes a pair of elastomeric clip tips respectively extending from the pair of lever arms. The pair of elastomeric clip tips are configured to grip the sterile drape when the catheter clip is clipped onto a sterile drape.

In some embodiments, the catheter clip further includes one or more pairs of stand-off tabs distributed between and extending from mating faces of the pair of lever arms. Each stand-off tab of the one-or-more pairs of stand-off tabs is offset from another stand-off tab of the one-or-more pairs of stand-off tabs, thereby establishing at least a portion of bilateral asymmetry in the catheter clip.

In some embodiments, the one-or-more pairs of stand-off tabs are placed longitudinally between the elastomeric clip tips and the elastomeric band such that the one-or-more pairs of stand-off tabs keep the elastomeric clip tips from fully contacting a rigid surface when the catheter clip is clipped onto the rigid surface. That said, the one-or-more pairs of stand-off tabs allow the elastomeric clip tips to grip the sterile drape when the catheter clip is clipped onto a sterile drape.

In some embodiments, the catheter retainer includes a base member and a socket extending from the base member. The base member is coupled to a mating face of a lever arm of the pair of lever arms. The socket is configured to accommodate insertion and removal of a proximal end portion of the catheter connector of the catheter.

In some embodiments, the base member is substantially annular with interlocking features including one or more notches in the base member, one or more projections extending from the base member, or some combination thereof. The interlocking features of the base member are configured to interlock with one or more complementary interlocking features in the mating face of the lever arm to which the base member of the catheter retainer is coupled. The interlocking features of the base member and the complementary interlocking features of the lever arm prevent rotation or any other movement of the base member relative to the lever arm to which the base member of the catheter retainer is coupled.

In some embodiments, at least one complementary interlocking feature of the one-or-more complementary interlocking features in the mating face of the lever arm to which the base member of the catheter retainer is coupled is configured with a guidewire through hole to pass an access guidewire therethrough. The complementary interlocking feature with the guidewire through hole thereby doubles as a guidewire support.

In some embodiments, the socket includes a plurality of deflectable members extending from a base of the socket. The deflectable members are configured to deflect away from a centerline axis of the socket for accommodating the insertion and removal of the proximal end portion of the catheter connector of the catheter.

In some embodiments, the socket includes a guidewire through hole through a base of the socket. The guidewire through hole of the socket is aligned with a centerline axis of the socket and the guidewire through hole of the complementary interlocking feature that doubles as the guidewire support. When the access guidewire is present, the access guidewire extends from the catheter connector of the catheter though both the guidewire through hole of the socket and the guidewire through hole of the complementary interlocking feature that doubles as the guidewire support.

In some embodiments, each lever arm of the pair of lever arms includes an opening in the handle end portion of the catheter clip. When the access guidewire is present, at least the opening in the lever arm to which the base member of the catheter retainer is coupled allows visualization of the access guidewire extending from the guidewire through hole of the socket to the guidewire through hole of the complementary interlocking feature that doubles as the guidewire support.

In some embodiments, the base member includes an adhesive configured to adhere the access guidewire to the base member when the access guidewire extends from the guidewire through hole of the socket.

Also disclosed herein is a catheter insertion assembly including, in some embodiments, a catheter, a catheter clip, and an access guidewire. The catheter clip is configured to clip onto a sterile drape that establishes at least a portion of a sterile field. The catheter clip includes a pair of lever arms mated to each other, an elastomeric band, and a catheter retainer. Each lever arm of the pair of lever arms is bilaterally asymmetric but identical to the other lever arm of the pair of lever arms. The elastomeric band is over a jaw end portion of the catheter clip. The catheter retainer is coupled to a handle end portion of the catheter clip. The catheter retainer is configured to hold a catheter connector of a catheter such that at least a proximal portion of the catheter remains in a sterile field when the catheter clip is clipped onto the sterile drape. The access guidewire includes a proximal portion and a distal portion. The proximal portion of the access guidewire is disposed in a primary lumen of the catheter but for a proximal end portion of the access guidewire extending from the catheter connector, through a socket of the catheter retainer, and over a base member of the catheter retainer to which the access guidewire is adhered.

In some embodiments, the catheter insertion assembly further includes an introducer needle and a coupler. The introducer needle includes a needle hub integral with a proximal portion of a composite shaft. The coupler couples the catheter and the introducer needle together by way of at least the access guidewire. The coupler includes a coupler housing and a valve module. The needle hub couples to the coupler housing in a ready-to-deploy state of the catheter insertion assembly. The valve module is disposed in the coupler housing, and both the composite shaft and the access guidewire pass through the valve module in the ready-to-deploy state of the catheter insertion assembly. The distal portion of the access guidewire is disposed in a needle lumen of the introducer needle in the ready-to-deploy state of the catheter insertion assembly.

Also disclosed herein is a method of forming a catheter clip. The method includes, in some embodiments, molding a pair of lever arms, each lever arm of the pair of lever arms being bilaterally asymmetric but identical to the other lever arm of the pair of lever arms. The method also includes molding a catheter retainer configured to hold a catheter connector of a catheter. The method also includes coupling the catheter retainer to a handle end portion of a lever arm of the pair of lever arms. The method also includes mating mating faces of the pair of lever arms to each other, thereby forming a nascent catheter clip. The method also includes disposing an elastomeric band over a jaw end portion of the nascent catheter clip, thereby forming the catheter clip. The catheter retainer is configured to hold the catheter connector of the catheter such that at least a proximal portion of the catheter remains in a sterile field when the catheter clip is clipped onto a sterile drape establishing at least a portion of the sterile field.

Also disclosed herein is a method of forming a catheter insertion assembly. The method includes, in some embodiments, obtaining at least a catheter, a catheter clip, and an access guidewire. The catheter clip includes a pair of lever arms mated to each other, an elastomeric band, and a catheter retainer. Each lever arm of the pair of lever arms is bilaterally asymmetric but identical to the other lever arm of the pair of lever arms. The elastomeric band is over a jaw end portion of the catheter clip. The catheter retainer is coupled to a handle end portion of the catheter clip. The method also includes inserting the access guidewire into a primary lumen of a catheter. The method also includes inserting a proximal end portion of the access guidewire through a guidewire through hole of a socket of the catheter retainer. The method also includes adhering the proximal end portion of the access guidewire over a base member of the catheter retainer. The method also includes inserting a proximal end portion of a catheter connector of the catheter into the socket of the catheter retainer. The catheter retainer is configured to hold the catheter connector of the catheter such that at least a proximal portion of the catheter remains in a sterile field when the catheter clip is clipped onto a sterile drape that establishes at least a portion of the sterile field.

Also disclosed herein is a method of using a catheter insertion assembly. The method includes, in some embodiments, establishing at least a portion of a sterile field about a patient with a sterile drape. The method also includes obtaining the catheter insertion assembly packaged in some packaging therefor. The catheter assembly includes an introducer needle, a coupler, a catheter, a catheter clip, and an access guidewire assembled together. The method also includes unclipping the catheter clip from a rib of a tray of the packaging in which the catheter insertion assembly is disposed in its packaged state. The catheter clip includes a pair of lever arms mated to each other, an elastomeric band, and a catheter retainer. Each lever arm of the pair of lever arms is bilaterally asymmetric but identical to the other lever arm of the pair of lever arms. The elastomeric band is over a jaw end portion of the catheter clip. The catheter retainer is coupled to a handle end portion of the catheter clip. The method also includes clipping the catheter onto the sterile drape. The catheter retainer holds a catheter connector of the catheter therein such that at least a proximal portion of the catheter remains in the sterile field for a venipuncture with the introducer needle of the catheter insertion assembly.

These and other features of the concepts provided herein will become more apparent to those of skill in the art in view of the accompanying drawings and following description, which describe particular embodiments of such concepts in greater detail.

### BRIEF DESCRIPTION OF DRAWINGS

FIG. 1 illustrates a catheter insertion assembly including an introducer needle, a coupler, a catheter, a catheter clip, and an access guidewire in accordance with some embodiments.

FIG. 2 illustrates the catheter insertion assembly with a tray of some packaging for the catheter insertion assembly in accordance with some embodiments.

FIG. 3 illustrates a detailed view of the catheter clip clipped onto a rib of the tray in accordance with some embodiments.

FIG. 4 illustrates another detailed view of the catheter clip clipped onto the rib of the tray in accordance with some embodiments.

FIG. 5 illustrates a detailed view of the catheter clip clipped onto a sterile drape in accordance with some embodiments.

FIG. 6 illustrates a detailed view of a catheter retainer of the catheter clip holding a catheter connector of the catheter in accordance with some embodiments.

FIG. 7 illustrates a detailed view of the catheter clip with the catheter connector of the catheter removed from the catheter retainer in accordance with some embodiments.

FIG. 8 illustrates a detailed view of the catheter clip without the catheter retainer in accordance with some embodiments.

FIG. 9 illustrates a detailed view of a lever arm of the catheter clip in accordance with some embodiments.

FIG. 10 illustrates a detailed view of the catheter retainer coupled to the lever arm of the catheter clip in accordance with some embodiments.

FIG. 11 illustrates another detailed view of the catheter retainer of the catheter clip holding the catheter connector of the catheter in accordance with some embodiments.

FIG. 12 illustrates a longitudinal cross section of the catheter retainer of the catheter clip holding the catheter connector of the catheter in accordance with some embodiments.

FIG. 13 illustrates a detailed end-on view of the catheter retainer of the catheter clip holding the catheter connector of the catheter in accordance with some embodiments.

FIG. 14 illustrates another detailed end-on view of the catheter retainer of the catheter clip holding the catheter connector of the catheter in accordance with some embodiments, albeit with the catheter connector rotated in the catheter retainer from that shown in FIG. 13.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

“Proximal” is used to indicate a portion, section, piece, element, or the like of a medical device intended to be near or relatively nearer to a clinician when the medical device is used on a patient. For example, a “proximal portion” or “proximal section” of the medical device includes a portion or section of the medical device intended to be near the clinician when the medical device is used on the patient. Likewise, a “proximal length” of the medical device includes a length of the medical device intended to be near the clinician when the medical device is used on the patient. A “proximal end” of the medical device is an end of the medical device intended to be near the clinician when the medical device is used on the patient. The proximal portion, the proximal section, or the proximal length of the medical device need not include the proximal end of the medical device. Indeed, the proximal portion, the proximal section, or the proximal length of the medical device can be short of the proximal end of the medical device. However, the proximal portion, the proximal section, or the proximal length of the medical device can include the proximal end of the medical device. Should context not suggest the proximal portion, the proximal section, or the proximal length of the medical device includes the proximal end of the medical device, or if it is deemed expedient in the following description, “proximal portion,” “proximal section,” or “proximal length” can be modified to indicate such a portion, section, or length includes an end portion, an end section, or an end length of the medical device for a “proximal end portion,” a “proximal end section,” or a “proximal end length” of the medical device, respectively.

“Distal” is used to indicate a portion, section, piece, element, or the like of a medical device intended to be near, relatively nearer, or even in a patient when the medical device is used on the patient. For example, a “distal portion” or “distal section” of the medical device includes a portion or section of the medical device intended to be near, relatively nearer, or even in the patient when the medical device is used on the patient. Likewise, a “distal length” of the medical device includes a length of the medical device intended to be near, relatively nearer, or even in the patient when the medical device is used on the patient. A “distal end” of the medical device is an end of the medical device intended to be near, relatively nearer, or even in the patient when the medical device is used on the patient. The distal portion, the distal section, or the distal length of the medical device need not include the distal end of the medical device. Indeed, the distal portion, the distal section, or the distal length of the medical device can be short of the distal end of the medical device. However, the distal portion, the distal section, or the distal length of the medical device can include the distal end of the medical device. Should context not suggest the distal portion, the distal section, or the distal length of the medical device includes the distal end of the medical device, or if it is deemed expedient in the following description, “distal portion,” “distal section,” or “distal length” can be modified to indicate such a portion, section, or length includes an end portion, an end section, or an end length of the medical device for a “distal end portion,” a “distal end section,” or a “distal end length” of the medical device, respectively.

Unless defined otherwise, all technical and scientific terms used herein have the same meaning as commonly understood by those of ordinary skill in the art.

As set forth above, CRBSIs lead to significant health and economic burdens, so sterile catheterization technique with CVCs is vital for preventing CRBSIs.

Disclosed herein are catheter clips and catheter insertion assemblies including such catheter clips that facilitate sterile catheterization technique. In an example, a catheter clip can include a pair of lever arms mated to each other. Each lever arm of the pair of lever arms can be bilaterally asymmetric but identical to the other lever arm of the pair of lever arms. An elastomeric band can be over a jaw end portion of the catheter clip. A catheter retainer can be coupled to a handle end portion of the catheter clip. The catheter retainer can be configured to hold a catheter connector of a catheter such that at least a proximal portion of the catheter remains in a sterile field when the catheter clip is clipped onto a sterile drape that establishes at least a portion of the sterile field.

### Catheter Insertion Assemblies

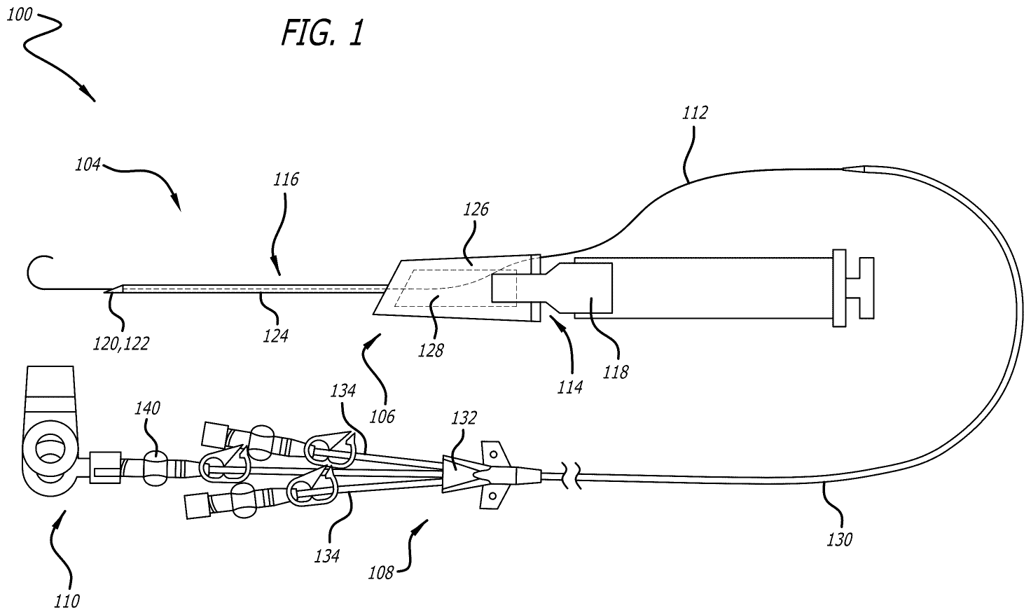

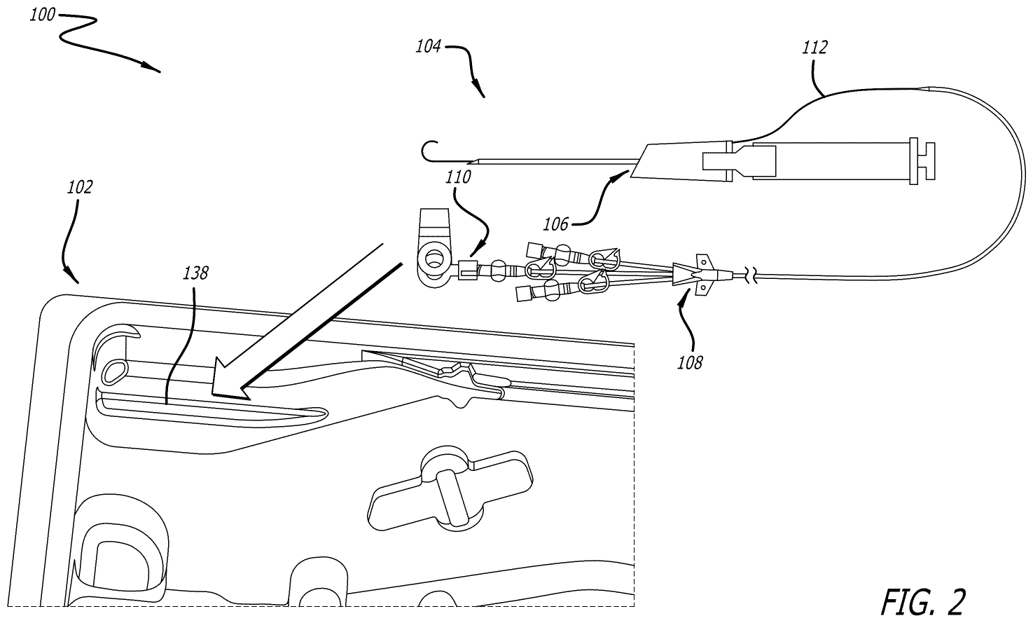

FIGS. 1 and 2 illustrate a catheter insertion assembly 100 in accordance with some embodiments. In addition to the catheter insertion assembly 100, FIG. 2 illustrates a tray 102 of some packaging for the catheter insertion assembly 100 in accordance with some embodiments.

As shown, the catheter insertion assembly 100 can include an introducer needle 104, a coupler 106, a catheter 108, a catheter clip 110, and an access guidewire 112 assembled together, the latter two components of which (i.e., the catheter clip 110 and the access guidewire 112) can be referred to herein as a catheter-clip assembly whether separate from or together with the catheter insertion assembly 100. In an example, the catheter insertion assembly 100 can include the catheter 108, the catheter clip 110, and the access guidewire 112 assembled together. In another example, the catheter insertion assembly 100 can include the introducer needle 104, the coupler 106, the catheter 108, the catheter clip 110, and the access guidewire 112 assembled together, wherein the coupler 106 couples the introducer needle 104 and the catheter 108 together by way of at least the access guidewire 112. Notably, as to the latter example, while a ‘J’-shaped distal tip of the access guidewire 112 is shown in FIGS. 1 and 2, the distal portion of the access guidewire 112 is withdrawn such that the distal tip of the access guidewire 112 is disposed in the introducer-needle lumen just proximal of the needle tip 122 of the introducer needle 104 in a ready-to-deploy state of the catheter insertion assembly 100. In this way, the distal tip of the access guidewire 112 can be immediately available to advance into a blood vessel lumen of a patient after a venipuncture with the introducer needle 104.

When present in the catheter insertion assembly 100, the introducer needle 104 includes a needle hub 114 integral with a proximal portion of a composite shaft 116, which needle hub 114 can include an integral needle-hub clip 118 configured to clip onto and, thereby, couple with the coupler 106 in the ready-to-deploy state of the catheter insertion assembly 100. While not shown in detail, the composite shaft 116 includes a needle shaft 120 (e.g., a stainless-steel needle shaft) having a longitudinal needle slot proximally extending from a heel of a bevel of a distal needle tip 122 of the needle shaft 120. The composite shaft 116 also includes a sheath 124 (e.g., a polymeric sheath) over the needle shaft 120 sealing the needle slot thereunder but for a sheath opening in a proximal portion of the sheath 124 through which sheath opening the access guidewire 112 passes into an introducer-needle lumen of the introducer needle 104 in the ready-to-deploy state of the catheter insertion assembly 100.

When present in the catheter insertion assembly 100, the coupler 106 includes a coupler housing 126 and a valve module 128. The coupler 106 housing can include external recesses in a proximal portion thereof, the external recesses configured to accept insertion of complementary clip protrusions of the needle-hub clip 118 of the needle hub 114 to keep at least the introducer needle 104 and the coupler 106 assembled together in the ready-to-deploy state of the catheter insertion assembly 100. The valve module 128 is disposed in the coupler 106 housing, and both the composite shaft 116 and the access guidewire 112 pass through the valve module 128 in the ready-to-deploy state of the catheter insertion assembly 100 such that the valve module 128 forms seals over both the composite shaft 116 and the access guidewire 112.

Notably, after a venipuncture with the introducer needle 104, the sheath 124 can be automatically cut along the longitudinal needle slot with an integrated blade integrated into the valve module 128 to allow the access guidewire 112 to escape from the introducer needle 104 during withdrawal of the introducer needle 104 from the coupler 106. Alternatively, at some time before withdrawal of the introducer needle 104 from the coupler 106, the sheath 124 can be manually cut along the longitudinal needle slot with a scalpel or the like to allow the access guidewire 112 to escape from the introducer needle 104. Like the longitudinal needle slot in the needle shaft 120 of the introducer needle 104, the coupler 106 can include a longitudinal coupler slot along its length to allow the access guidewire 112 to also escape from the coupler 106.

As to the catheter 108, the catheter 108 includes a catheter tube 130, a catheter hub 132, and one or more extension legs 134 coupled together in the foregoing order. Indeed, the catheter tube 130 includes a proximal end portion disposed in the catheter hub 132, and each extension leg of the one-or-more extension legs 134 includes a distal end portion disposed in the catheter hub 132. Such a catheter 108 can be a central venous catheter (“CVC”), as shown; however, it should be understood the catheter 108 is not limited thereto, as the catheter 108 can be any catheter intended for placement in a blood vessel lumen of a patient.

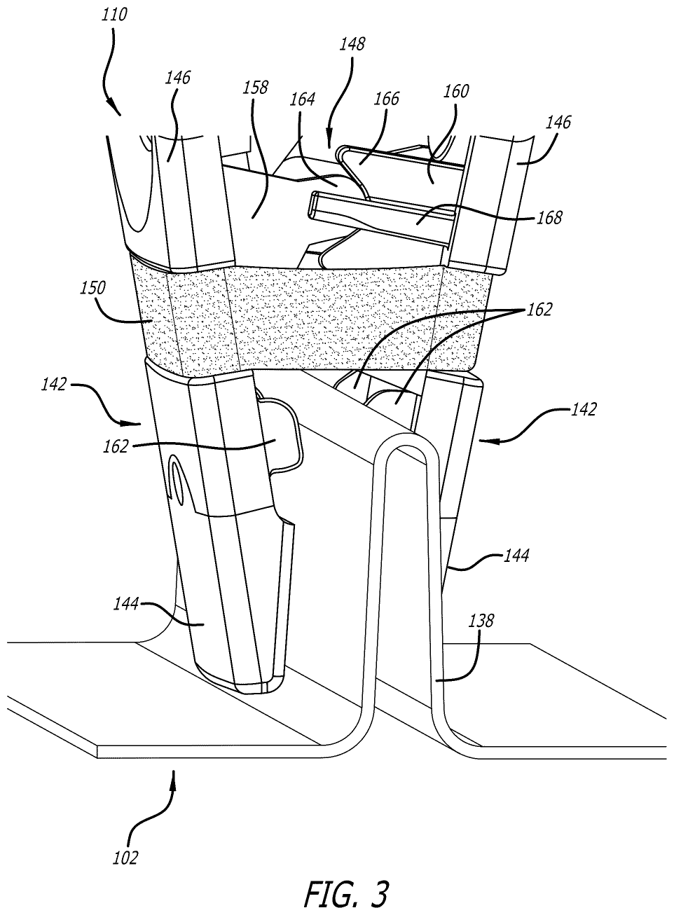

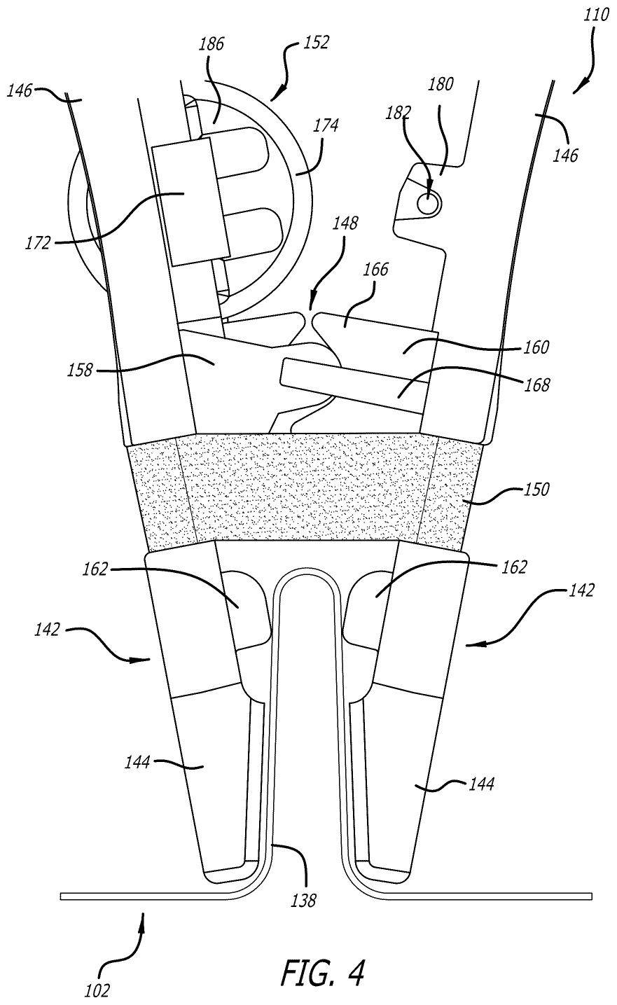

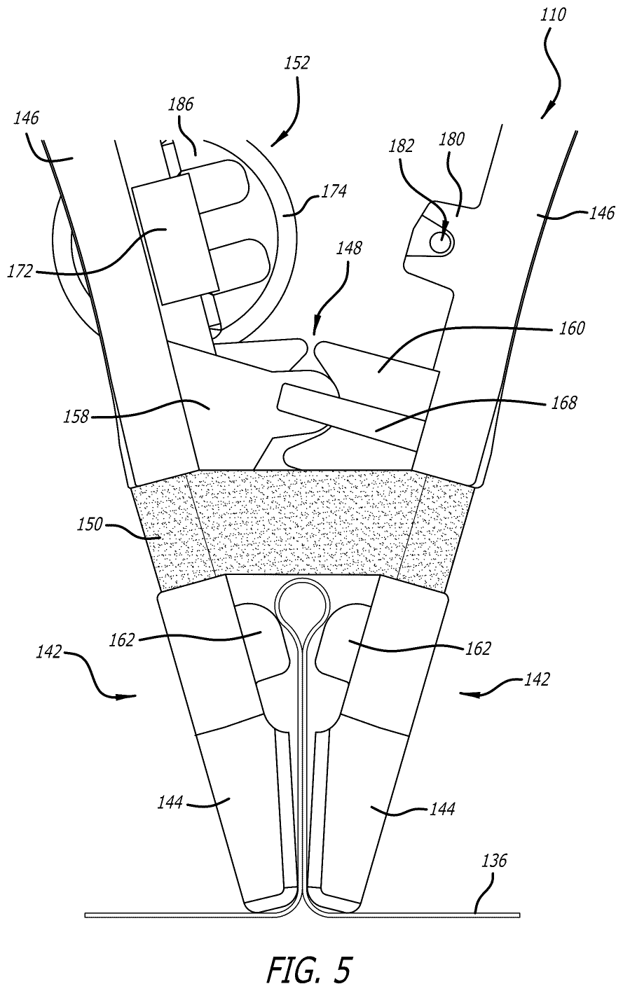

As to the catheter clip 110, the catheter clip 110 is configured to keep at least the catheter 108 and the access guidewire 112 assembled together in the ready-to-deploy state of the catheter insertion assembly 100. Further, the catheter clip 110 is configured to clip onto a substrate to effectuate one or more other objectives. In an example, the catheter clip 110 is configured to clip onto a sterile drape 136 that establishes at least a portion of a sterile field for a medical procedure (e.g., catheterization). (See FIG. 5.) When the catheter clip 110 is part of the catheter insertion assembly 100 and the catheter clip 110 is clipped onto the sterile drape 136, the catheter clip 110 is thusly configured to hold the catheter 108 by the catheter connector 140 such that at least a proximal portion of the catheter 108 remains in the sterile field during the medical procedure. Being that the proximal portion of the access guidewire 112 is adhered to the catheter retainer 152 in the ready-to-deploy state of the catheter insertion assembly 100, as set forth below, and being that a remainder of the access guidewire 112 is disposed in the primary lumen of the catheter 108 in the ready-to-deploy state of the catheter insertion assembly 100, the catheter clip 110 is thusly also configured to keep the proximal portion of the access guidewire 112 in the sterile field during the medical procedure. In another example, the catheter clip 110 is configured to clip onto a rigid surface such as a rib 138 of the tray 102 of some packaging in which the catheter insertion assembly 100 packaged. (See FIGS. 3 and 4.) When the catheter clip 110 is part of the catheter insertion assembly 100 and the catheter clip 110 is clipped onto the rib 138 of the tray 102, the catheter clip 110 is thusly configured to hold the catheter 108 by the catheter connector 140 such that the catheter insertion assembly 100 is in a fixed location and orientation that a clinician can expect upon removing outer packaging of the packaging in which the catheter insertion assembly 100 packaged, thereby providing another element of routine in the medical procure that can help conserve cognitive resources and, thus, lead to better medical outcomes. Additional description for the catheter clip 110 is set forth below in the separate section thereon.

The access guidewire 112 includes a proximal portion and a distal portion. In the ready-to-deploy state of the catheter insertion assembly 100, the proximal portion of the access guidewire 112 is disposed in a primary lumen of the catheter 108 but for a proximal end portion of the access guidewire 112. Indeed, the proximal end portion of the access guidewire 112 extends from a catheter connector 140 (e.g., a Luer connector), through the socket 174 of the catheter retainer 152, and over the base member 172 of the catheter retainer 152 to which the access guidewire 112 is adhered.

### Catheter Clips

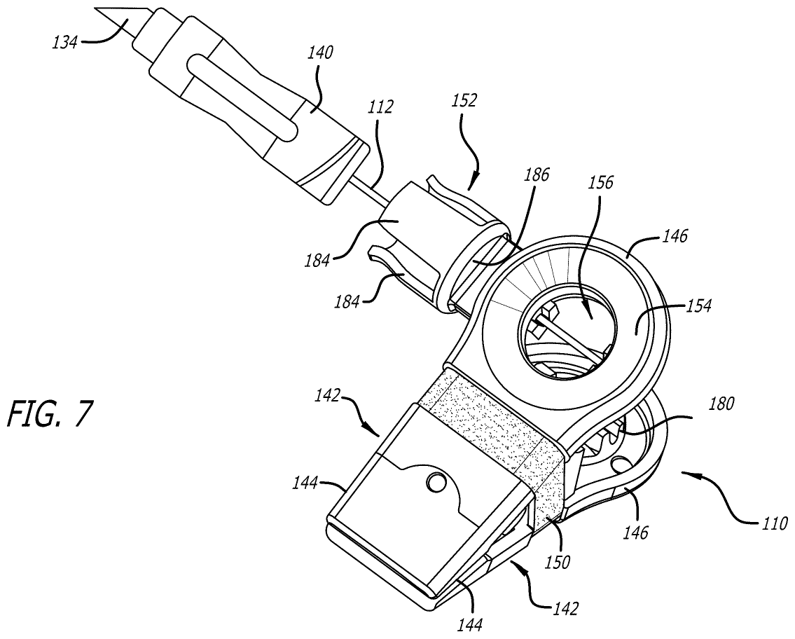

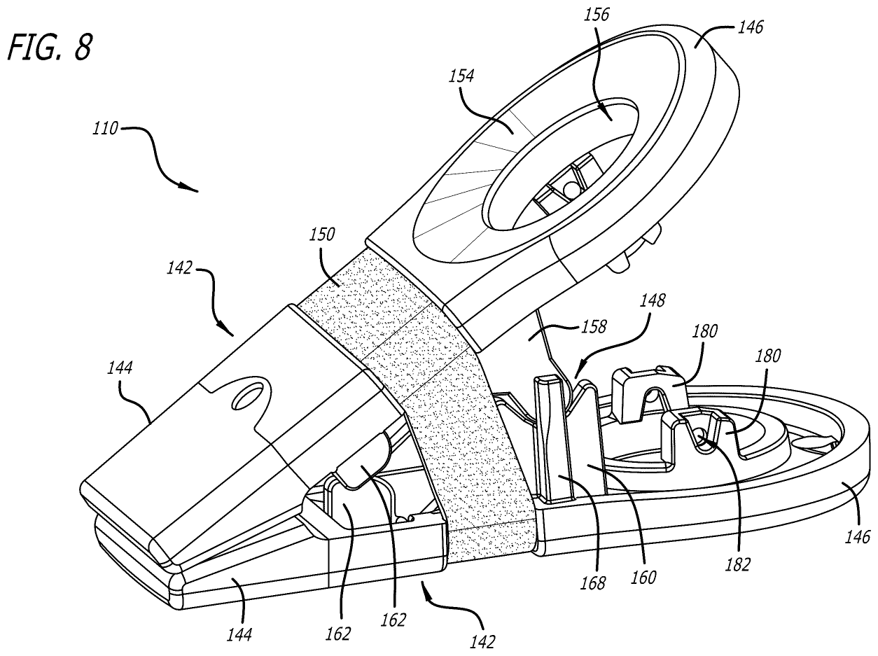

FIGS. 3-14 various views of the catheter clip 110 in accordance with some embodiments.

As shown, the catheter clip 110 can include a pair of lever arms 142 mated to each other forming a pair of clip tips 144, a pair of handles 146, and a shared pair of fulcrum points 148 between mating faces of the pair of lever arms 142. Further, the catheter clip 110 can include an elastomeric band 150 over the pair of lever arms 142 and a catheter retainer 152 coupled to a lever arm of the pair of lever arms 142. Notably, the catheter clip 110 can have longitudinal portions including at least a jaw portion and a handle portion, which portions are generally opposite each other, the portions being distributed across the pair of fulcrum points 148 set forth below. When such longitudinal portions include their corresponding longitudinal ends, the longitudinal portions are referred to herein as longitudinal end portions. Thus, the catheter clip 110 can also have longitudinal end portions including at least a jaw end portion and a handle end portion, which end portions are generally opposite each other, the end portions being distributed across the pair of fulcrum points 148 set forth below. In an example, the elastomeric band 150 can be over a jaw portion or jaw end portion of the catheter clip 110, and the catheter retainer 152 can be coupled to a handle portion or handle end portion of the catheter clip 110.

The pair of clip tips 144 can be a pair of elastomeric clip tips in the jaw end portion of the catheter clip 110 respectively extending from the pair of lever arms 142. Such a pair of clip tips 144 can be respectively molded over the pair of lever arms 142, the pair of clip tips 144 configured to grip a sterile drape such as the sterile drape 136 when the catheter clip 110 is clipped onto the sterile drape 136 as shown in FIG. 5. Indeed, the pair of elastomeric clips tips are configured with a tackiness to grip the sterile drape 136 when the catheter clip 110 is clipped onto the sterile drape 136. However, such tackiness can make it difficult to remove the catheter clip 110 from a rigid surface such as the catheter-clip counterpiece of the tray 102. For this reason, the catheter clip 110 can include the pairs of stand-off tabs 162 set forth below keep the clip tips 144 from contacting, substantially contacting, or fully contacting the rigid surface when the catheter clip 110 is clipped onto the rigid surface.

The pair of handles 146 can include a pair of bowl-shaped depressions 154 in the handle end portion of the catheter clip 110 respectively molded into the pair of lever arms 142. Further, each lever arm of the pair of lever arms 142 can include an opening 156 in its handle end portion, the opening 156 through the bowl-shaped depression 154 of the lever arm 142. The pair of bowl-shaped depressions 154 in the handle end portion of the catheter clip 110 together with their respective openings 156 serve as finger grips in that fingertip flesh can slightly insert into the openings 156 for gripping the pair of handles 146 while squeezing the handle end portion of the catheter clip 110 to open it. Notably, when the access guidewire 112 is present such as in the catheter-clip assembly or the catheter insertion assembly 100, at least the opening 156 in the lever arm 142 to which the base member 172 of the catheter retainer 152 is coupled allows visualization of the access guidewire 112 extending from the guidewire through hole 198 of the socket 174 to the guidewire through hole 182 of the complementary interlocking feature (e.g., the abutment 180) that doubles as the guidewire support.

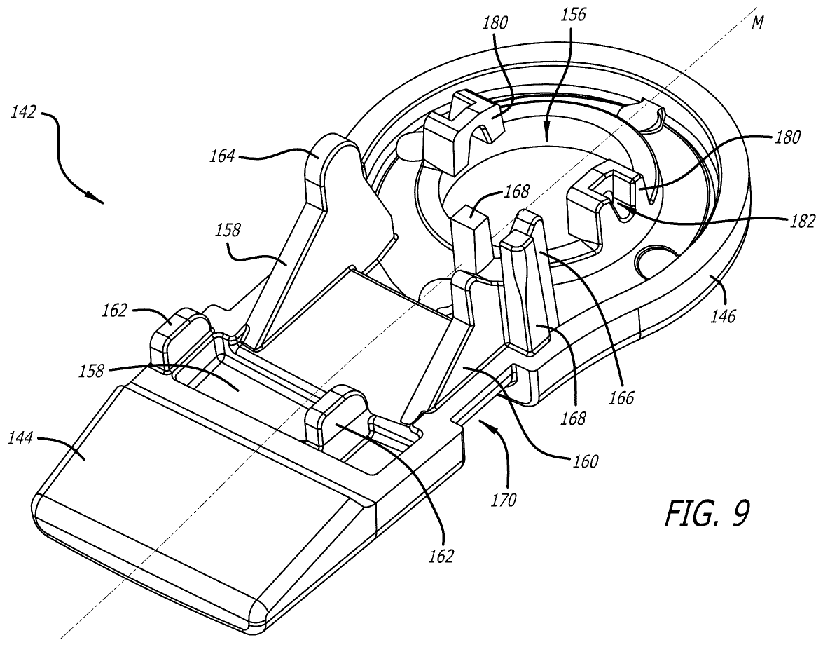

Each lever arm of the pair of lever arms 142 can be bilaterally asymmetric but identical to the other lever arm of the pair of lever arms 142. Indeed, each lever arm of the pair of lever arms 142 can include both an axle extension tab 158 and a bearing extension tab 160 extending from a mating face thereof. The axle extension tab 158 and the bearing extension tab 160 can establish at least a portion of bilateral asymmetry in the catheter clip 110 as shown in FIG. 9 by way of a lever-arm midline M. Further, each lever arm of the pair of lever arms 142 can include one or more stand-off tabs 162 extending from the mating face thereof. When there is one stand-off tab 162 extending from the mating face of each lever arm of the pair of lever arms 142, the stand-off tab 162 can be offset in terms of its transverse distance from the lever-arm midline M akin to that shown in FIG. 9, thereby establishing at least a portion of the bilateral asymmetry in the catheter clip 110. When there are two or more stand-off tabs 162 extending from the mating face of each lever arm of the pair of lever arms 142, each stand-off tab of the two-or-more stand-off tabs 162 can be offset from another stand-off tab of the two-or-more stand-off tabs 162 in terms of its transverse distance from the lever-arm midline M as shown in FIG. 9, thereby establishing at least a portion of the bilateral asymmetry in the catheter clip 110. Manufacturing of the catheter clip 110 from molding pieces of a single bilaterally asymmetric lever arm 142 to assembling the catheter clip 110 by mating the mating faces of such pieces is thereby advantageously simplified.

The pair of fulcrum points 148 between the mating faces of the pair of lever arms 142 can be defined by a discontinuous axle distributed in a corresponding discontinuous bearing over a width of the catheter clip 110. Indeed, an axle portion 164 of the axle extension tab 158 of each lever arm of the pair of lever arms 142 can be seated in an opposing bearing portion 166 of the bearing extension tab 160 of the other lever arm of the pair of lever arms 142, thereby establishing the discontinuous axle distributed in the corresponding discontinuous bearing over the width of the catheter clip 110. Notably, the bearing extension tab 160 of each lever arm of the pair of lever arms 142 can include one or two side posts 168 lateral of the bearing portion 166 of the bearing extension tab 160. The one-or-two side posts 168 are configured to keep the axle portion 164 of the axle extension tab 158 seated in the bearing portion 166 of the bearing extension tab 160.

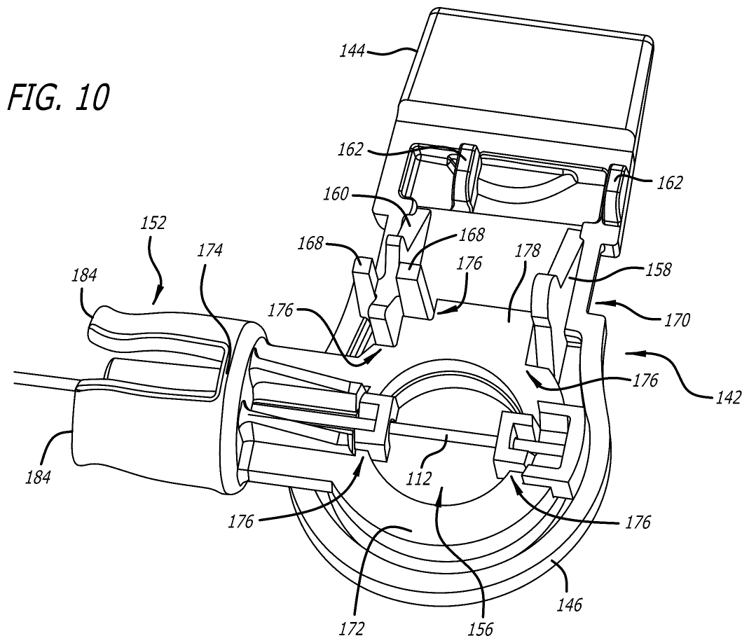

Again, each lever arm of the pair of lever arms 142 can include one or more stand-off tabs 162 extending from the mating face thereof as shown in FIGS. 9 and 10. As to the catheter clip 110 on the whole, the catheter clip 110 can include one or more pairs of stand-off tabs 162 distributed between and extending from the mating faces of the pair of lever arms 142 as shown in FIGS. 3-6 and 8. In an example, each lever arm of the pair of lever arms 142 can include one stand-off tab 162 extending from the mating face thereof for one pair of stand-off tabs 162 distributed between the mating faces of the pair of lever arms 142. In another example, each lever arm of the pair of lever arms 142 can include two stand-off tabs 162 extending from the mating face thereof for two pairs of stand-off tabs 162 distributed between the mating faces of the pair of lever arms 142 as best shown in FIG. 3. Each stand-off tab of the one-or-more pairs of stand-off tabs 162 can be offset from another stand-off tab of the one-or-more pairs of stand-off tabs 162, thereby establishing at least a portion of the bilateral asymmetry in the catheter clip 110. That said, in some embodiments, each stand-off tab of the one-or-more pairs of stand-off tabs 162 can be aligned with another stand-off tab of the one-or-more pairs of stand-off tabs 162. While there might be some bilateral symmetry in each lever arm of the pair of lever arms 142 with respect to the stand-off tabs 162 in such embodiments, each lever arm of the pair of lever arms 142 remains bilaterally asymmetric on account of at least the axle extension tab 158 and the bearing extension tab 160.

When each lever arm of the pair of lever arms 142 includes the two stand-off tabs 162 extending from the mating face thereof for the two pairs of stand-off tabs 162 distributed between the mating faces of the pair of lever arms 142, which, again, is best shown in FIG. 3, each lever arm-based pair of stand-off tabs 162 (as opposed to some pair of stand-off tabs 162 distributed between the pair of lever arms 142) can be offset from another lever arm-based pair of stand-off tabs 162 in the catheter clip 110 such that the lever arm-based pairs of stand-off tabs 162 interleave when the catheter clip 110 is at least partially closed. Such pairs of stand-off tabs 162, as with all stand-off tabs 162 disclosed herein, are placed longitudinally between the clip tips 144 and the elastomeric band 150 or, alternatively, the clip tips 144 and the axle and bearing extension tabs 158 and 160, such that the lever arm-based pairs of stand-off tabs 162 keep the clip tips 144 from contacting, substantially contacting, or fully contacting a rigid surface (e.g., the rib 138 of the tray 102) when the catheter clip 110 is clipped onto the rigid surface. Indeed, provided the rigid surface is inserted far enough into the catheter clip 110 for interaction with the lever arm-based pairs of stand-off tabs 162, the rigid surface bridges each pair of the lever arm-based pair of stand-off tabs 162, as shown in FIGS. 3 and 4, preventing the lever arm-based pairs of stand-off tabs 162 from interleaving and, thereby, preventing the clip tips 144 from contacting, substantially contacting, or fully contacting the rigid surface. As shown in FIG. 5, however, the lever arm-based pairs of stand-off tabs 162 allow the clip tips 144 to fully contact and grip the sterile drape 136 when the catheter clip 110 is clipped onto the sterile drape 136. This is due to the sterile drape 136 being flexible, foldable, or the like, which allows the lever arm-based pairs of stand-off tabs 162 to interleave with the sterile drape 136 therebetween.

Notably, as to the tray 102 and the rib 138 thereof for clipping the catheter clip 110 thereon, the rib 138 can be more generally referred to as a catheter-clip counterpiece of the tray 102 or the packaging in which the catheter insertion assembly 100 is packaged. The catheter-clip counterpiece can be molded together with the tray 102 in a unitary tray, or the catheter-clip counterpiece can be separately molded from the tray 102 and, optionally, assembled therewith for an assembled tray. If the catheter-clip counterpiece is separately molded from the tray 102, the catheter-clip counterpiece can be disposed in a catheter compartment of the tray 102 and allowed to freely move therein, in which case it can be referred to as a free catheter-clip counterpiece. Further, if the catheter-clip counterpiece is separately molded from the tray 102, the catheter-clip counterpiece can alternatively be adhered to or snapped into the catheter compartment of the tray 102 and, thereby, secured therein for a secured catheter-clip counterpiece. Whether the catheter-clip counterpiece is free in the catheter compartment of the tray 102 or secured therein, the catheter-clip counterpiece can have a height or an equivalent dimension for a corresponding shape of the catheter-clip counterpiece (e.g., a diameter for a disk-shaped catheter-clip counterpiece) ranging from just enough for sufficient interaction with the stand-off tabs 162 distributed between the pair of lever arms 142 of the catheter clip 110 to commensurate with a height of one or more walls of the catheter compartment. Lastly, the catheter-clip centerpiece can have a flange akin to that of a ‘T’-beam used in construction, particularly in embodiments in which the catheter clip 110 does not include any of the stand-off tabs 162.

An external face of each lever arm of the pair of lever arms 142 can include a transverse channel 170 configured to accommodate the elastomeric band 150 therein. With respect to each lever arm of the pair of lever arms 142, the channel 170 can be longitudinally located between the elastomeric clip tip 144 and the axle and bearing extension tabs 158 and 160 of the lever arm 142. With respect to the catheter clip 110 on the whole, the channel 170 can be longitudinally located between the pair of clip tips 144 and the pair of fulcrum points 148. The elastomeric band 150 is thusly offset from the pair of fulcrum points 148 toward the pair of clip tips 144, thereby causing the catheter clip 110 to remain closed in a default, closed state of the catheter clip 110 with the pair of clip tips 144 in contact with each other. That said, in some embodiments, the channel 170 can be longitudinally located closer to the pair of fulcrum points 148 than that shown. The channel 170 can even extend to the pair of fulcrum points 148 or past the pair of fulcrum points 148 such that the channel 170 is longitudinally located over the pair of fulcrum points 148 to some extent. In an example, a handle end-most wall of the channel 170 can be over the pair of fulcrum points 148 such that a handle end-most edge of the elastomeric band 150 is likewise over the pair of fulcrum points 148. In another example, the handle end-most wall of the channel 170 can extend past the pair of fulcrum points 148 such that the handle end-most edge of the elastomeric band 150 likewise extends past the pair of fulcrum points 148. When the handle end-most edge of the elastomeric band 150 is over the pair of fulcrum points 148 or extends past them, a portion of the elastomeric band 150 can extend past the pair of fulcrum points 148 when the handle end portion of the catheter clip 110 is squeezed to effectuate an opened state of the catheter clip 110 in which the pair of clip tips 144 no longer contact each other. This advantageously creates a balance in elastomeric-band tension between the opened and closed states of the catheter clip 110, thereby requiring less squeezing force to hold open the catheter clip 110 when it is already in the opened state.

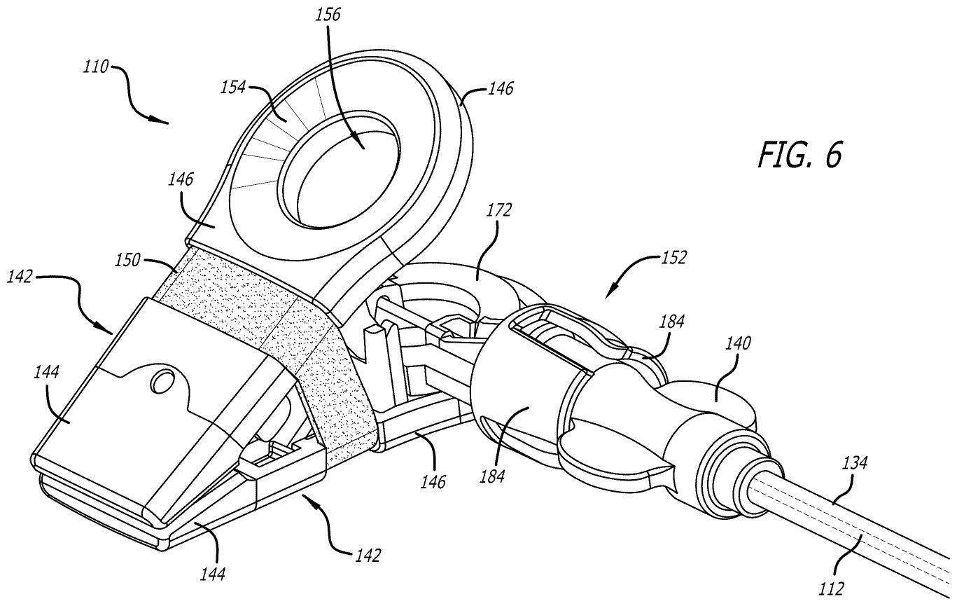

Again, the catheter retainer 152 can be coupled to a lever arm of the pair of lever arms 142 as shown in FIGS. 6 and 10. The catheter retainer 152 can be configured to hold the catheter connector 140 of the catheter 108 such that at least the proximal portion of the catheter 108 remains in a sterile field when the catheter clip 110 is clipped onto a sterile drape (e.g., the sterile drape 136) that establishes at least a portion of the sterile field. That, and the catheter retainer 152 being configured to hold the catheter connector 140 of the catheter 108 also fixes the catheter insertion assembly 100 in a fixed location and orientation in the tray 102 of the packaging in which the catheter insertion assembly 100 is packaged where a clinician can expect the catheter insertion assembly 100 upon removing the outer packaging around the tray 102.

The catheter retainer 152 can include a base member 172 and a socket 174 extending from the base member 172. While the socket 174 can extend from the base member 172 orthogonally with respect to the pair of clip tips 144 as shown in FIG. 6, which is a convenient orientation between the socket 174 and the pair of clip tips 144 for clipping the catheter clip 110 onto a sterile drape (e.g., the sterile drape 136) about a patient and subsequently introducing the catheter 108 into the patient, the socket 174 can extend from the base member 172 directly away from the pair of clip tips 144 of the catheter clip 110 in an alternative orientation between the socket 174 and the pair of clip tips 144. That said, the socket 174 can extend from the base member 172 with any desired orientation between the socket 174 and the pair of clip tips 144 that does not decrease venipuncture ergonomics, promote kinking of the catheter 108 or the access guidewire 112, or the like.

The base member 172 of the catheter retainer 152 can be coupled to the mating face of a lever arm of the pair of lever arms 142 in the catheter clip 110. Indeed, the base member 172 can be adhered with an adhesive to the mating face of the foregoing lever arm 142. Such an adhesive can be a same adhesive as that configured to adhere the access guidewire 112 to the base member 172 such as the distal end portion of the access guidewire 112 that extends from the guidewire through hole 198 of the socket 174 onto the base member 172, each abutment of the pair of abutments 180 set forth below, and again onto the base member 172 as shown in FIG. 10. In fact, application of the adhesive to adhere the access guidewire 112 to the base member 172 and, optionally, each abutment of the pair of abutments 180, during assembly can result in excess adhesive dripping down between the base member 172 and corresponding lever arm 142, thereby also adhering the base member 172 to the mating face of the foregoing lever arm 142. Notably, being that the access guidewire 112 passes through at least the complementary interlocking feature of the lever arm 142 to which the base member 172 of the catheter retainer 152 is coupled, as set forth below, the access guidewire 112 itself, through interference, can also couple the base member 172 to the mating face of the lever arm of the pair of lever arms 142 in the catheter clip 110.

Structurally, the base member 172 of the catheter retainer 152 is substantially annular with interlocking features including one or more notches 176 in the base member 172, one or more projections 178 extending from the base member 172, or some combination thereof. The interlocking features of the base member 172 are configured to interlock with one or more complementary interlocking features in the mating face of the lever arm 142 to which the base member 172 of the catheter retainer 152 is coupled. As shown in FIG. 10, the interlocking features of the base member 172 can include at least a pair of notches 176 disrupting annularity of an outer perimeter of the base member 172, wherein the pair of notches 176 are configured to accommodate and interlock with the axle and bearing extension tabs 158 and 160 extending from the lever arm 142 to which the base member 172 of the catheter retainer 152 is coupled, the axle and bearing extension tabs 158 and 160 doubling as the complementary interlocking features of the lever arm 142. In another example, the interlocking features of the base member 172 can include a projection 178 extending from the substantially annular base member 172, wherein the projection 178 is configured to occupy space between the axle and bearing extension tabs 158 and 160 extending from the lever arm 142 to which the base member 172 of the catheter retainer 152 is coupled, thereby interlocking with the axle and bearing extension tabs 158 and 160, which, again, double as the complementary interlocking features of the lever arm 142. Notably, the foregoing projection 178, itself, can include one of the notches 176 configured to accommodate and interlock with an internal side post 168 lateral of the bearing portion 166 of the bearing extension tab 160, the foregoing side post 168 of the bearing extension tab 160 doubling as the complementary interlocking features of the lever arm 142. Such interlocking features of the base member 172 and the complementary interlocking features of the lever arm 142 mitigate any externally applied forces and, thereby, prevent rotation or any other movement of the base member 172 relative to the lever arm 142 to which the base member 172 of the catheter retainer 152 is coupled.

However, the interlocking features of the base member 172 of the catheter retainer 152 are not limited to interlocking in some way with the complementary interlocking features of the axle and bearing extension tabs 158 and 160 of the lever arm 142 to which the base member 172 of the catheter retainer 152 is coupled. Indeed, the interlocking features of the base member 172 can include up to at least a pair of notches 176 disrupting annularity of an inner perimeter of the base member 172, wherein the pair of notches 176 are configured to accommodate and interlock with up to at least a pair of abutments 180 extending from the mating face on opposite sides of the opening 156 of the lever arm 142 to which the base member 172 of the catheter retainer 152 is coupled. Notably, each abutment of the pair of abutments 180 can include a guidewire through hole 182 to pass the access guidewire 112 therethrough such that the access guidewire 112 bridges across the pair of abutments 180 and the opening 156 in the handle end portion of the foregoing lever arm 142 that is flanked by the pair of abutments 180. The pair of abutments 180 thereby function as a guidewire support across the foregoing opening 156 and double as the complementary interlocking features of the lever arm 142.

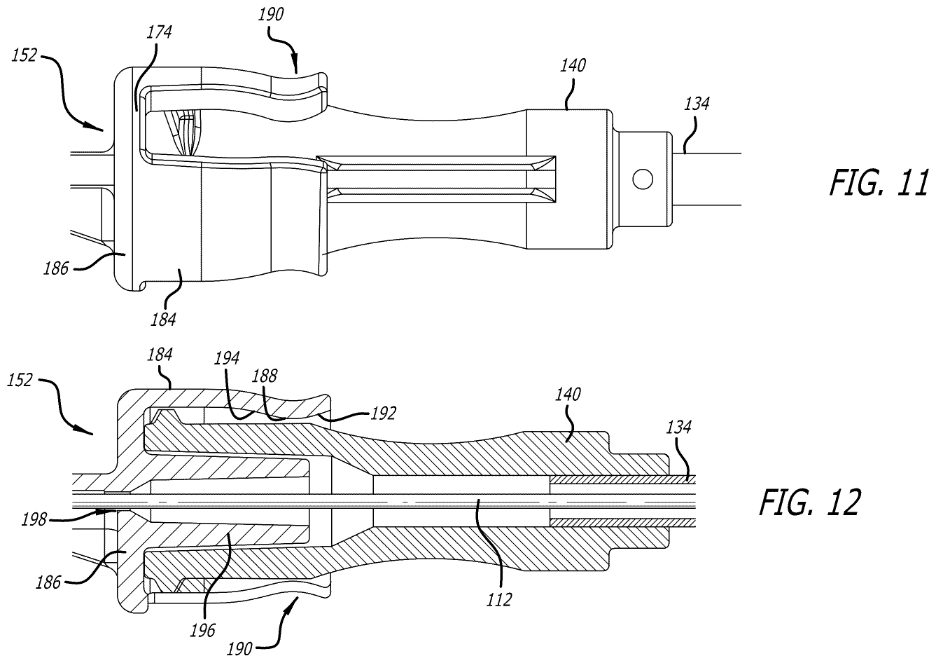

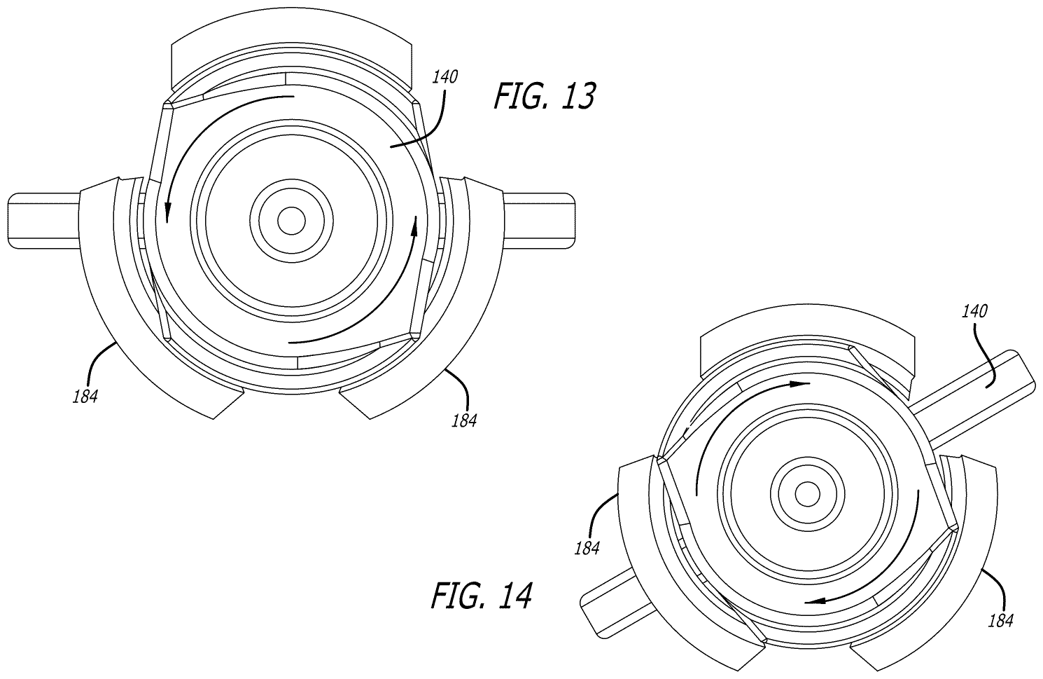

As shown in FIGS. 11-14, the socket 174 of the catheter retainer 152 can be configured to accommodate insertion and removal of a proximal end portion of the catheter connector 140 of the catheter 108. Indeed, the socket 174 can include a plurality of deflectable members 184 such as two, three, four, or more deflectable members 184 extending from a base 186 of the socket 174 in a circumferential arrangement about a centerline axis of the socket 174. The deflectable members 184 can be configured to deflect away from the centerline axis of the socket 174 to accommodate the insertion and removal of the proximal end portion of the catheter connector 140 of the catheter 108. Notably, each deflectable member of the deflectable members 184 can include an arcuate protrusion 188 in an interior face of the deflectable member 184 facing the centerline axis of the socket 174 and, optionally, a corresponding arcuate depression 190 in an opposite, exterior face of the deflectable member 184, for at least a plurality of arcuate protrusions 188 that form a constriction around the socket 174 having a smaller inner diameter than an outer diameter of the catheter connector 140 of the catheter 108 including any external threads (e.g., double-start threads) thereof. However, each deflectable member of the deflectable members 184 can also include a lead-in lip 192 in the interior face of the deflectable member 184 leading in to the arcuate protrusion 188 such that a plurality of such lead-in lips 192 form a mouth around the socket 174 having at least a commensurate inner diameter commensurate with the outer diameter of the catheter connector 140 of the catheter 108. Further, each deflectable member of the deflectable members 184 can also include a lead-out portion 194 in the interior face of the deflectable member 184 leading out to the arcuate protrusion 188 such that a plurality of such lead-out portions 194 lead out to the constriction around the socket 174. In this way, the deflectable members 184 can deflect when the catheter connector 140 is inserted into the mouth formed by the lead-in lips 192 around the socket 174, the constriction around the socket 174 can hold the catheter connector 140 in the socket 174 akin to a detent, and the deflectable members 184 can again deflect when the catheter connector 140 is removed from the socket 174 by way of the lead-out portions 194 around the socket 174. Advantageously, the deflectable members 184 are sized and shaped to the catheter connector 140 and any external threads thereof such that all of the deflectable members 184 deflect at a same time during the insertion of the catheter connector 140, rotation of the catheter connector 140 as shown in FIGS. 13 and 14, and removal of the catheter connector 140 of the catheter 108 from the socket 174 of the catheter retainer 152.

As shown in FIG. 12, the socket 174 of the catheter retainer 152 can include a centering unit 196 and a guidewire through hole 198 through both the centering unit 196 and the base 186 of the socket 174 if the centering unit 196 is indeed present. The guidewire through hole 198 of the socket 174 can be aligned with both the centerline axis of the socket 174 and the guidewire through hole 182 of each abutment 180 present in the catheter clip 110, which abutments 180, as set forth above, can function as both guidewire supports and complementary interlocking features of the lever arm 142. When the access guidewire 112 is present in the catheter insertion assembly 100, such as in the ready-to-deploy state of the catheter insertion assembly 100, the access guidewire 112 extends from the catheter connector 140 of the catheter 108 though the guidewire through hole 198 of the socket 174 and through the guidewire through holes 182 of any abutments 180 of the catheter clip 110.

### Methods

Methods include at least a method of forming the catheter clip 110, a method of forming the catheter insertion assembly 100, and a method of using the catheter insertion assembly 100, which includes a method of using the catheter clip 110.

As to the method of forming the catheter clip 110, the method includes molding the pair of lever arms 142, wherein each lever arm of the pair of lever arms 142 is bilaterally asymmetric but identical to the other lever arm of the pair of lever arms 142. The method also includes molding the catheter retainer 152, which, as set forth above, is configured to hold the catheter connector 140 of the catheter 108. The method also includes coupling the catheter retainer 152 to the handle end portion of the lever arm of the pair of lever arms 142. The method also includes mating the mating faces of the pair of lever arms 142 to each other, thereby forming a nascent catheter clip. The method also includes disposing the elastomeric band 150 over the jaw end portion of the nascent catheter clip, thereby forming the catheter clip 110.

As to the method of forming the catheter insertion assembly 100, the method includes obtaining at least the catheter 108, the catheter clip 110, and the access guidewire 112. The method also includes inserting the access guidewire 112 into the primary lumen of the catheter 108. The method also includes inserting the proximal end portion of the access guidewire 112 through the guidewire through hole 198 of the socket 174 of the catheter retainer 152. The method also includes adhering the proximal end portion of the access guidewire 112 over the base member 172 of the catheter retainer 152. The method also includes inserting the proximal end portion of the catheter connector 140 of the catheter 108 into the socket 174 of the catheter retainer 152. The method also includes coupling the introducer needle 104 and the catheter 108 together by way of the access guidewire 112 through the coupler 106.

As to the method of using the catheter insertion assembly 100, the method includes establishing at least a portion of a sterile field about a patient with a sterile drape such as the sterile drape 136. The method also includes obtaining the catheter insertion assembly 100 packaged in some packaging therefor. The method also includes unclipping the catheter clip 110 from the rib 138 of the tray 102 of the packaging in which the catheter insertion assembly 100 is disposed in its packaged state. The method also includes clipping the catheter 108 onto the sterile drape 136. Again, the catheter retainer 152 holds the catheter connector 140 of the catheter 108 therein such that at least the proximal portion of the catheter 108 remains in the sterile field for a venipuncture with the introducer needle 104 of the catheter insertion assembly 100.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

What is claimed is:

1. A catheter clip, comprising:
a pair of lever arms mated to each other, each lever arm of the pair of lever arms being bilaterally asymmetric but identical to the other lever arm of the pair of lever arms;
an elastomeric band over a jaw end portion of the catheter clip;
a catheter retainer coupled to a handle end portion of the catheter clip, the catheter retainer configured to hold a catheter connector of a catheter such that at least a proximal portion of the catheter remains in a sterile field when the catheter clip is clipped onto a sterile drape establishing at least a portion of the sterile field.

2. The catheter clip of claim 1, further comprising a shared pair of fulcrum points between mating faces of the pair of lever arms, the pair of fulcrum points defined by a discontinuous axle distributed in a discontinuous bearing over a width of the catheter clip.

3. The catheter clip of claim 2, wherein each lever arm of the pair of lever arms includes both an axle extension tab and a bearing extension tab extending from a mating face thereof, thereby establishing at least a portion of bilateral asymmetry in the catheter clip.

4. The catheter clip of claim 3, wherein an axle portion of the axle extension tab of each lever arm of the pair of lever arms is seated in an opposing bearing portion of the bearing extension tab of the other lever arm of the pair of lever arms, thereby establishing the discontinuous axle distributed in the discontinuous bearing over the width of the catheter clip.

5. The catheter clip of claim 4, wherein the bearing extension tab of each lever arm of the pair of lever arms includes one or two side posts lateral of the bearing portion of the bearing extension tab, the one-or-two side posts configured to keep the axle portion of the axle extension tab seated in the bearing portion of the bearing extension tab.

6. The catheter clip of claim 2, wherein an external face of each lever arm of the pair of lever arms includes a transverse channel longitudinally located between the pair of fulcrum points and a pair of elastomeric clip tips respectively extending from the pair of lever arms in the jaw end portion of the catheter clip, the channel of each lever arm of the pair of lever arms configured to accommodate the elastomeric band therein.

7. The catheter clip of claim 1, wherein the jaw end portion of the catheter clip includes a pair of elastomeric clip tips respectively extending from the pair of lever arms, the pair of elastomeric clip tips configured to grip the sterile drape when the catheter clip is clipped onto a sterile drape.

8. The catheter clip of claim 7, further comprising one or more pairs of stand-off tabs distributed between and extending from mating faces of the pair of lever arms, each stand-off tab of the one-or-more pairs of stand-off tabs offset from another stand-off tab of the one-or-more pairs of stand-off tabs, thereby establishing at least a portion of bilateral asymmetry in the catheter clip.

9. The catheter clip of claim 8, wherein the one-or-more pairs of stand-off tabs are placed longitudinally between the elastomeric clip tips and the elastomeric band such that the one-or-more pairs of stand-off tabs keep the elastomeric clip tips from fully contacting a rigid surface when the catheter clip is clipped onto the rigid surface but allow the elastomeric clip tips to grip the sterile drape when the catheter clip is clipped onto a sterile drape.

10. The catheter clip of claim 1, wherein the catheter retainer includes a base member and a socket extending from the base member, the base member coupled to a mating face of a lever arm of the pair of lever arms, and the socket configured to accommodate insertion and removal of a proximal end portion of the catheter connector of the catheter.

11. The catheter clip of claim 10, wherein the base member is substantially annular with interlocking features including one or more notches in the base member, one or more projections extending from the base member, or some combination thereof, the interlocking features of the base member configured to interlock with one or more complementary interlocking features in the mating face of the lever arm to which the base member of the catheter retainer is coupled, thereby preventing rotation or any other movement of the base member relative to the lever arm to which the base member of the catheter retainer is coupled.

12. The catheter clip of claim 11, wherein at least one complementary interlocking feature of the one-or-more complementary interlocking features in the mating face of the lever arm to which the base member of the catheter retainer is coupled is configured with a guidewire through hole to pass an access guidewire therethrough, the complementary interlocking feature with the guidewire through hole thereby doubling as a guidewire support.

13. The catheter clip of claim 12, wherein the socket includes a plurality of deflectable members extending from a base of the socket, the deflectable members configured to deflect away from a centerline axis of the socket for accommodating the insertion and removal of the proximal end portion of the catheter connector of the catheter.

14. The catheter clip of claim 12, wherein the socket includes a guidewire through hole through a base of the socket, the guidewire through hole of the socket aligned with a centerline axis of the socket and the guidewire through hole of the complementary interlocking feature that doubles as the guidewire support such that, when the access guidewire is present, the access guidewire extends from the catheter connector of the catheter though both the guidewire through hole of the socket and the guidewire through hole of the complementary interlocking feature that doubles as the guidewire support.

15. The catheter clip of claim 14, wherein each lever arm of the pair of lever arms includes an opening in the handle end portion of the catheter clip, at least the opening in the lever arm to which the base member of the catheter retainer is coupled allowing, when the access guidewire is present, visualization of the access guidewire extending from the guidewire through hole of the socket to the guidewire through hole of the complementary interlocking feature that doubles as the guidewire support.

16. The catheter clip of claim 15, wherein the base member includes an adhesive configured to adhere the access guidewire to the base member when the access guidewire extends from the guidewire through hole of the socket.

17. A catheter insertion assembly, comprising:
a catheter;
a catheter clip configured to clip onto a sterile drape establishing at least a portion of a sterile field, the catheter clip including:
a pair of lever arms mated to each other, each lever arm of the pair of lever arms being bilaterally asymmetric but identical to the other lever arm of the pair of lever arms;
an elastomeric band over a jaw end portion of the catheter clip; and
a catheter retainer coupled to a handle end portion of the catheter clip, the catheter retainer configured to hold a catheter connector of the catheter such that at least a proximal portion of the catheter remains in the sterile field when the catheter clip is clipped onto the sterile drape; and
an access guidewire including a proximal portion and a distal portion, the proximal portion of the access guidewire disposed in a primary lumen of the catheter but for a proximal end portion of the access guidewire extending from the catheter connector, through a socket of the catheter retainer, and over a base member of the catheter retainer to which the access guidewire is adhered.

18. The catheter insertion assembly of claim 17, further comprising:
an introducer needle including a needle hub integral with a proximal portion of a composite shaft; and
a coupler coupling the catheter and the introducer needle together by way of at least the access guidewire, the coupler including:
a coupler housing to which the needle hub couples in a ready-to-deploy state of the catheter insertion assembly; and
a valve module disposed in the coupler housing through which both the composite shaft and the access guidewire pass, the distal portion of the access guidewire disposed in a needle lumen of the introducer needle in the ready-to-deploy state of the catheter insertion assembly.

19. A method of forming a catheter clip, comprising:
molding a pair of lever arms, each lever arm of the pair of lever arms being bilaterally asymmetric but identical to the other lever arm of the pair of lever arms;
molding a catheter retainer configured to hold a catheter connector of a catheter;
coupling the catheter retainer to a handle end portion of a lever arm of the pair of lever arms;
mating mating faces of the pair of lever arms to each other, thereby forming a nascent catheter clip; and
disposing an elastomeric band over a jaw end portion of the nascent catheter clip, thereby forming the catheter clip, the catheter retainer configured to hold the catheter connector of the catheter such that at least a proximal portion of the catheter remains in a sterile field when the catheter clip is clipped onto a sterile drape establishing at least a portion of the sterile field.

20. A method of forming a catheter insertion assembly, comprising:
obtaining at least a catheter, a catheter clip, and an access guidewire, the catheter clip including:
a pair of lever arms mated to each other, each lever arm of the pair of lever arms being bilaterally asymmetric but identical to the other lever arm of the pair of lever arms;
an elastomeric band over a jaw end portion of the catheter clip;
a catheter retainer coupled to a handle end portion of the catheter clip, inserting the access guidewire into a primary lumen of a catheter;
inserting a proximal end portion of the access guidewire through a guidewire through hole of a socket of the catheter retainer;
adhering the proximal end portion of the access guidewire over a base member of the catheter retainer; and
inserting a proximal end portion of a catheter connector of the catheter into the socket of the catheter retainer, the catheter retainer configured to hold the catheter connector of the catheter such that at least a proximal portion of the catheter remains in a sterile field when the catheter clip is clipped onto a sterile drape establishing at least a portion of the sterile field.

21. A method of using a catheter insertion assembly, comprising:
establishing at least a portion of a sterile field about a patient with a sterile drape;
obtaining the catheter insertion assembly packaged in some packaging therefor, the catheter assembly including an introducer needle, a coupler, a catheter, a catheter clip, and an access guidewire assembled together;
unclipping the catheter clip from a rib of a tray of the packaging in which the catheter insertion assembly is disposed in its packaged state, the catheter clip including:
a pair of lever arms mated to each other, each lever arm of the pair of lever arms being bilaterally asymmetric but identical to the other lever arm of the pair of lever arms;
an elastomeric band over a jaw end portion of the catheter clip; and
a catheter retainer coupled to a handle end portion of the catheter clip; and
clipping the catheter onto the sterile drape, the catheter retainer holding a catheter connector of the catheter therein such that at least a proximal portion of the catheter remains in the sterile field for a venipuncture with the introducer needle of the catheter insertion assembly.
